(19) United States
(12) Patent Application Publication
(10) Pub. No.: US 2019/0186115 A1
(43) Pub. Date: Jun. 20, 2019

(54) Bariatric Bidet Seat System

(71) Applicant: The Mountain Group LLC, Storrs, CT (US)

(72) Inventors: Brian Schwab, East Chatham, NY (US); Shaoyu Peng, Huatan Township (TW)

(21) Appl. No.: 15/847,119

(22) Filed: Dec. 19, 2017

(51) Int. Cl. E03D 9/08; A47K 10/48; A47K 17/02; A47K 13/24; A61M 3/06

(52) U.S. Cl. CPC E03D 9/08; A47K 10/48; A61M 3/06; A47K 17/026; A47K 13/24; A47K 17/02

(57) ABSTRACT

Aspects of the present invention provide a method and a toilet seat system for bariatric users. The bariatric toilet seat system includes a toilet, a bidet seat assembly, and one or more bidet-support spring mechanisms, a flange support mechanism, a lumbar support mechanism, an arm mechanism, and combinations thereof. Heavy weights of a bariatric user is better distributed or absorbed by the design of the bariatric toilet seat system. The bariatric toilet seat system can be automated with a user-friendly control system built inside the bariatric toilet seat system and controlled by an attached control unit or a remote control. Methods of forming the bariatric toilet seat system and methods using the bariatric toilet seat system are also disclosed.

### BACKGROUND OF THE INVENTION

Embodiments of the present invention generally relate to toilet sear apparatus and system can be used to wash and clean a region of a human body. More specifically, aspects of the present invention provide a method and apparatus for delivering and applying water, medication, and/or a cleaning solution to a region of the body (e.g., genital or anal area, intimate parts, perianal region) of a heavy weight user who may have difficulty to access and drying the region.

### DESCRIPTION OF THE RELATED ART

Bidets and other modern toilet seat systems have been used to spray water and clean private parts of a user while using a toilet. The bidet systems are used for washing the genital and anal areas using cleaning water of appropriate temperature sprayed from the center of the bidet system, instead of a toilet paper after relief of stool or urination. Originally being developed for washing the pubic area for females, bidet systems have has now been popular among people of all ages and both sexes because it is known to be more hygienic to wash the intimate parts and anus with water instead of paper after relief. In addition, cleansing the pubic/anal regions with water may help to avoid infection and prevent hemorrhoids and other anal disease. Furthermore, it is very effective for women with gynecology diseases. It is also very useful for the elderly or obese people to relieve themselves with great convenience.

However, most toilets are incapable of supporting bariatric (i.e., overweight) users. Conventional toilets have a seat designed to receive each user's weight and distributed onto the toilet when in use. When a bariatric individual using a toilet exceeds the toilet's support capacity, significant problems may occur. In addition, most toilets with bidet seat cleaning systems are not designed for supporting a bariatric user, either.

Many wall-mounted toilets have problems in supporting a bariatric user, thus often being broken off the wall due to over-weight. In addition, floor-mounted toilets are known to collapse after use by a bariatric user. The use of a conventional toilet by a bariatric user often results in serious injuries when the toilet collapses, including, lacerations to the buttocks, thighs, and back. Further, the terrible discomfort and embarrassment after such incident should not be underestimated.

In addition, most bariatric seats do not include any bidets systems, because of design incompatibility, in-supportability, and other design and installment problems. However, bariatric users have much greater needs to use the cleaning and drying mechanisms of bidets and bidet seat systems because they often have difficulty in reaching out to their genital or anal area, intimate parts, and perianal regions to clean themselves after the use of toilet. Therefore, there is a need for designing a novel bariatric bidet seat system that can support most bariatric users and help them wash and clean their genital and/or anal area.

### SUMMARY OF THE INVENTION

The present invention generally includes a method and a toilet seat system for delivering water, medicine, washing, cleaning, and drying a region of a human body of a bariatric human subject. The toilet seat system includes a bidet seat assembly, which comprises a drying assembly, a spraying nozzle assembly, and/or a medicine delivery assembly, among others.

In one embodiment, the toilet seat system for supporting a body of a human subject includes, a toilet, one or more bidet-support spring mechanisms positioned adjacent to the toilet, and a bidet seat assembly being positioned atop the one or more bidet-support spring mechanisms and the toilet to support the body of the human subject, wherein the one or more bidet-support spring mechanisms are connected to a bottom of a seat body of the bidet seat assembly and provided to support the bidet seat assembly so that the bidet seat assembly is capable of being flexibly movable in vertical direction.

In one aspect, the bidet seat assembly comprises a spraying nozzle assembly and/or a drying assembly, where the drying assembly is provided for blowing out air at a temperature ranging from between room temperature to about 90 degree Celsius, and the spray nozzle assembly is provided for spraying out water at a temperature ranging from between room temperature to about 90 degree Celsius.

In still another aspect, the spray nozzle assembly may further includes a spraying nozzle, a first motor being connected to the spraying nozzle of the spray nozzle assembly, the first motor being capable of directing one or more extending and retracting movements of the spraying nozzle of the spray nozzle assembly, one or more second motors being connected to the spraying nozzle of the spray nozzle assembly and capable of directing one or more three-dimensional rotational movements of the spraying nozzle of the spray nozzle assembly, and one or more dynamic steering remote control units adapted to be communicating with both the first motor and the one or more second motors and directing one or more movements of the first motor and the one or more second motors. In still another aspect, the bidet seat assembly also includes a medicine delivery assembly for delivering medicine to the body of the human subject.

In another embodiment, the one or more bidet-support spring mechanisms of the toilet seat system include an attachment element to be attached to a rim of a toilet bowl of the toilet. The one or more bidet-support spring mechanisms of the toilet seat system may also include a spring connected to the attachment element so that the attachment element is flexibly movable in vertical direction to support the bidet seat assembly. Further, the one or more bidet-support spring mechanisms includes a support body, which is capable of being curved in an angle α, ranging vertically from zero degree to less than 90°.

In still another embodiment, the toilet seat system also includes a flange support mechanism having a support body and a flange floor support being secured to the floor by a number of screws. In one example, the flange support mechanism is adapted to be connected to the one or more bidet-support spring mechanisms and being secured to a floor where the toilet seat system is positioned thereon. In another example, the flange support mechanism of the toilet seat system includes a coupling element to couple a lumber support mechanism to the flange support mechanism.

In a further embodiment, the toilet seat system further includes a lumber support mechanism having a back support and a support body, where the arm is coupled to a flange support mechanism. In a still further embodiment, the toilet seat system includes one or more arm mechanisms, each arm mechanism including an armrest, one or more support bodies, where each armrest is provided to support an arm of the human subject. In one aspect, the one or more arm mechanisms are coupled to a flange support mechanism.

One or more embodiment of the invention also provides a toilet seat system for supporting a body of a human subject, including a toilet, one or more bidet-support spring mechanisms positioned adjacent to the toilet, a flange support mechanism connected to the one or more bidet-support spring mechanisms and being secured to a floor where the toilet seat system is positioned thereon, and a bidet seat assembly being positioned atop the one or more bidet-support spring mechanisms and the toilet to support the body of the human subject, wherein the one or more bidet-support spring mechanisms is connected to a bottom of a seat body of the bidet seat assembly and provided to support the bidet seat assembly so that the bidet seat assembly is capable of being flexibly movable in vertical direction. In one aspect, the bidet seat assembly includes a spray nozzle assembly for spraying out water at a temperature ranging from between room temperature to about 90 degree Celsius. In another aspect, the flange support mechanism of the toilet seat system includes a support body and a flange floor support being secure to the floor by a number of screws.

In one example, the flange support mechanism Is coupled to a lumber support mechanism. In another example, the flange support mechanism and the lumber support mechanism are coupled by a coupling element of the flange support mechanism. In still another example, the flange support mechanism is coupled to one or more arm mechanisms, where each of the one or more arm mechanisms includes one or more support bodies and an armrest, and each armrest is provided to support an arm of the human subject.

Another embodiment of the invention provides a toilet seat system including a toilet, one or more bidet-support spring mechanisms positioned adjacent to the toilet, a flange support mechanism connected to the one or more bidet-support spring mechanisms and being secured to a floor where the toilet seat system is positioned thereon, and a bidet seat assembly being positioned atop the one or more bidet-support spring mechanisms and the toilet to support the body of the human subject, where the one or more bidet-support spring mechanisms is connected to a bottom of a seat body of the bidet seat assembly and provided to support the bidet seat assembly so that the bidet seat assembly is capable of being flexibly movable in vertical direction, and the bidet seat assembly comprises a spray nozzle assembly for jetting out water at a temperature ranging from between room temperature to about 90 degree Celsius. In one example, the bidet seat assembly also includes a drying assembly for blowing out air at a temperature ranging from between room temperature to about 90 degree Celsius. In another example, the bidet seat assembly further includes a medicine delivery assembly for delivering medicine to the body of the human subject. In one aspect, the spray nozzle assembly includes a spraying nozzle, a first motor being connected to the spraying nozzle of the spray nozzle assembly, the first motor being capable of directing one or more extending and retracting movements of the spraying nozzle of the spray nozzle assembly, one or more second motors being connected to the spraying nozzle of the spray nozzle assembly and capable of directing one or more three-dimensional rotational movements of the spraying nozzle of the spray nozzle assembly, and one or more dynamic steering remote control units adapted to be communicating with both the first motor and the one or more second motors and directing one or more movements of the first motor and the one or more second motors.

One or more additional embodiments of the invention provide a method of producing a toilet seat system. In one embodiment, the method includes positioning one or more bidet-support spring mechanisms of the toilet seat system to be adjacent to a toilet, attaching an attachment element of the bidet-support spring mechanisms to a rim of a toilet bowl of the toilet, and securing the bidet-support spring mechanisms to a floor where the toilet seat system is positioned thereon. In one aspect, the method also include compressing a spring which is connected to the attachment element of one or more bidet-support spring mechanisms of the toilet seat system so that the attachment element is flexibly movable in vertical direction to support the bidet seat assembly. In anther embodiment, the method further includes curving a support body of the one or more bidet-support spring mechanisms in an angle α, ranging vertically from zero degree to less than 90°.

In another embodiment, the method may also include positioning a bidet seat assembly atop the one or more bidet-support spring mechanisms and the toilet to support a body of a human subject, and connecting the one or more bidet-support spring mechanisms to a bottom of a seat body of the bidet seat assembly to support the bidet seat assembly so that the bidet seat assembly is capable of being flexibly movable in vertical direction. In one aspect, the bidet seat assembly includes a spray nozzle assembly for jetting out water at a temperature ranging from between room temperature to about 90° C. In another aspect, the bidet seat assembly also includes a drying assembly for blowing out air at a temperature ranging from between room temperature to about 90° C. In a further aspect, the bidet seat assembly includes a medicine delivery assembly for delivering medicine to the body of the human subject. In one example, the spray nozzle assembly includes a spraying nozzle, a first motor being connected to the spraying nozzle of the spray nozzle assembly, the first motor being capable of directing one or more extending and retracting movements of the spraying nozzle of the spray nozzle assembly, one or more second motors being connected to the spraying nozzle of the spray nozzle assembly and capable of directing one or more three-dimensional rotational movements of the spraying nozzle of the spray nozzle assembly, and one or more dynamic steering remote control units adapted to be communicating with both the first motor and the one or more second motors and directing one or more movements of the first motor and the one or more second motors.

In still another embodiment, the method may further include connecting the one or more bidet-support spring mechanisms to a flange support mechanism. In one aspect, the method may also include coupling one or more arm mechanisms to the flange support mechanism, each arm mechanism having an armrest, one or more support bodies, where each armrest is provided to support an arm of the human subject. In yet still another embodiment, the method may further include coupling a lumber support mechanism to the flange support mechanism. The lumber support mechanism may include a back support and a support body coupled to the flange support mechanism.

Once a human object is sitting on the toilet seat system, a method of using the toilet seat system may include operating the bidet seat assembly and spraying water and/or medicine on a person's body (e.g., a bottom, genital or anus area of a human user). The method may further include using water or liquid to rinse the entire area of the person's bottom and cleaning with a cleaner, such as an enzymatic cleaner, and removing all matters that needed to be removed from the perineum. In one example, a barrier spray is applied. Further, the method may include using a drying assembly (e.g., a drying assembly with a timed high power fan) to blow dry an entire region or area of a genital or anus portion of a human user. The drying mechanism is provided to dry very fast, protect the skin, and promote wound care healing. All the above steps can be automated. The above are all done with a control unit (e.g., a remote control or a control unit installed on the front or the side of the bidet seat assembly).

Accordingly, a bariatric bidet seat system and a method of securing the bariatric bidet toilet seat system are provided herein to be able to securely support a number of bariatric human users and help them wash and clean their genital and/or anal area. The system and method are easy to be connected to auxiliary parts, and easy to install and use for washing and cleaning a region of a human body.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments. The drawings are not necessarily to scale. The drawings are merely schematic representations, not intended to portray specific parameters of the invention.

FIG. 1 is a perspective view of an example of a bariatric bidet toilet system having a toilet, one or more bidet-support spring mechanisms, a flange support mechanism, a lumbar support mechanism, an arm mechanism, and a bidet seat assembly according to embodiments of the invention.

FIG. 2 is an exploded view of the break-down of a bariatric toilet seat system having a toilet, a bidet seat assembly, two bidet-support spring mechanisms, two arm mechanisms, one lumbar support mechanism, and one flange support mechanism according to embodiments of the invention.

FIG. 3 is a perspective view of a lumber support mechanism having a back support and a support body according to one embodiment of the invention.

FIG. 4 is a perspective view of a flange support mechanism having a flange floor support, a support body, and a coupling element according to one embodiment of the invention.

FIG. 5A is a perspective view of one example of a bidet-support spring mechanism having an attachment element, and a support body according to one or more embodiments of the invention.

FIG. 5B is a perspective view of another example of a bidet-support spring mechanism, showing an attachment element, a spring, and a support body according to one or more embodiments of the invention.

FIG. 6 is a side view of a toilet seat assembly positioned atop a bidet-support spring mechanism and a toilet, showing a spring of the bidet-support spring mechanism is adapted to be flexibly moving up and down in a vertical direction “Z”, a support body of the bidet-support spring mechanism is curved at an angle α, such that the support body can moved in a direction “A”, according to one or more embodiments of the invention.

FIG. 7A is a perspective view of one example of an arm mechanism having an armrest, an upper support body, a lower support body, according to one or more embodiments of the invention.

FIG. 7B is a perspective view of another example of an arm mechanism according to one or more embodiments of the invention.

FIG. 8A provides a side view of one example of a bariatric toilet seat system where an armrest of an arm mechanism of the system is adapted to moved up in a direction “H” when the arm mechanism is not being pressed with a force, or is leveled with an up force according to one or more embodiments of the invention.

FIG. 8B provides a side view of another example of a bariatric toilet seat system where an armrest of an arm mechanism of the system is adapted to moved down in a direction “I” when the arm mechanism is being pressed with a down force according to one or more embodiments of the invention.

FIG. 9 is a perspective view of one example of a bariatric bidet toilet seat system where one or more bidet-support spring mechanisms are positioned to be connected to a flange support mechanism and be attached to a toilet, a toilet seat assembly is positioned atop the bidet-support spring mechanism, and one or more arm mechanisms are coupled to the one or more bidet-support spring mechanisms, according to one or more embodiments of the invention.

FIG. 10A is an enlarged side view of a bidet seat assembly having a seat cover covering a bidet seat above a case in a closed position, according to embodiments of the invention.

FIG. 10B is a perspective view of an example of a bidet seat assembly having a seat cover in an open position, a drying assembly, a spray nozzle assembly with a spraying nozzle, a base (optionally, with an medicine delivery assembly), a remote control unit, and a control unit adjacent the bidet seat assembly, according to embodiments of the invention.

FIG. 11A is a side view of one example of a bidet seat assembly positioned in an upward-relaxed position atop a bidet-support spring mechanism where the support body of the bidet-support spring mechanism is curved in a “P1” position, according to one or more embodiments of the invention.

FIG. 11B is a side view of another example of a bidet seat assembly positioned in a downward-compressed position atop a bidet-support spring mechanism where the support body of the bidet-support spring mechanism is curved in a “P2” position, according to one or more embodiments of the invention.

FIG. 11C is a side view of one example of a bariatric bidet toilet seat system having an arm mechanism, a lumber support mechanism, and a bidet seat assembly positioned in a downward-compressed position atop a bidet-support spring mechanism where the support body of the bidet-support spring mechanism is curved in a “P2 position, according to one or more embodiments of the invention.

FIG. 11D is a side view of another example of a bariatric bidet toilet seat system having an arm mechanism, a lumber support mechanism, and a bidet seat assembly positioned in an upward-relaxed position atop a bidet-support spring mechanism where the support body of the bidet-support spring mechanism is curved in a P1 position, according to one or more embodiments of the invention.

FIG. 11E illustrates one example when a human subject is using a bariatric bidet toilet seat system with a bidet seat assembly, a bidet-support spring mechanism, and sitting on top of the bidet seat assembly, according to one or more embodiments of the invention.

FIG. 12A is a side view of one example of a bariatric bidet toilet seat system having a lumber support mechanism, and a bidet seat assembly, where a cover of the bidet seat assembly is closed, according to one or more embodiments of the invention.

FIG. 12B is a side view of another example of a bariatric bidet toilet seat system having a lumber support mechanism, and a bidet seat assembly, where a cover of the bidet seat assembly is open and can be pressed against a back support of the lumber support mechanism so that the back support of the lumber support mechanism is in a position S1, according to one or more embodiments of the invention.

FIG. 12C is a side view of another example of a bariatric bidet toilet seat system having a lumber support mechanism, and a bidet seat assembly, where a cover of the bidet seat assembly is open, a human subject is sitting atop of the bidet seat assembly and leaning backward against a back support of the lumber support mechanism so that the back support of the lumber support mechanism is in a position S2, according to one or more embodiments of the invention.

FIG. 13 illustrates one example when a human subject on top of the bidet seat assembly after using a toilet bariatric bidet toilet seat system with hands pressing down an armrest of an arm mechanism, according to one or more embodiments of the invention.

### DETAILED DESCRIPTION

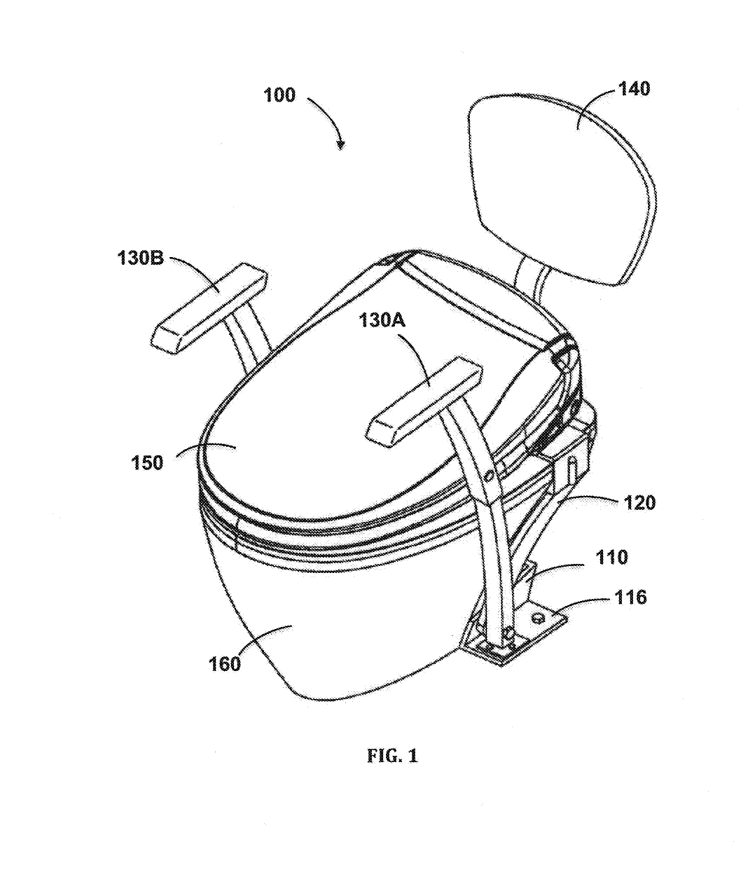

The present invention generally includes a method and a toilet seat system for bariatric used. The bariatric toilet seat system includes a toilet, a bidet seat assembly, and one or more bidet-support spring mechanisms, a flange support mechanism, a lumbar support mechanism, an arm mechanism, and combinations thereof. FIG. 1 is a perspective view of one example of a toilet seat system 100 for bariatric users. The toilet seat system 100 includes a toilet 160, a bidet seat assembly 150, a lumbar support mechanism 140, one or more arm mechanisms 130A, 130B, one or more bidet-support spring mechanisms 120, and a flange support mechanism 110 and according to embodiments of the invention.

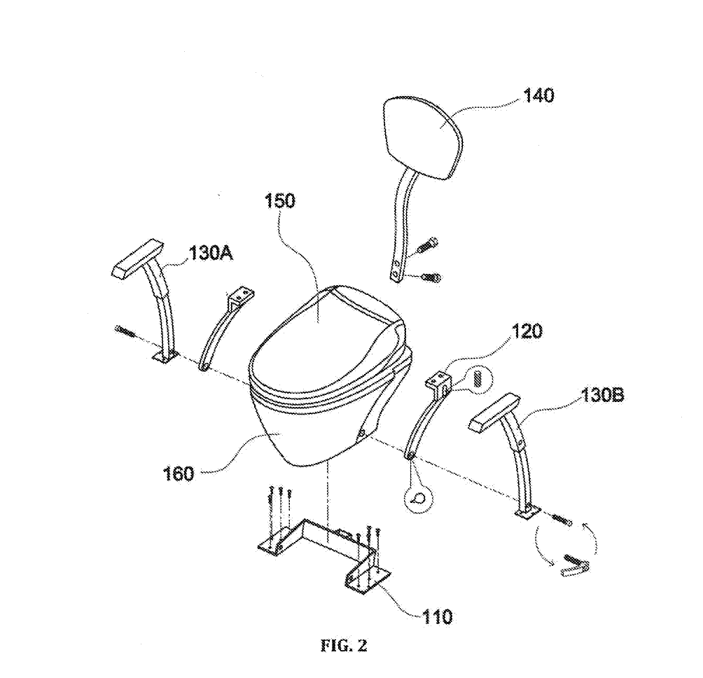

FIG. 2 is an exploded view of the break-down of various parts of one example of the toilet seat system 100, showing how its parts are connected and assembled together. For example, the toilet seat system 100 includes one toilet 160, one bidet seat assembly 150, one flange support mechanism 110 positioned near the bottom back portion of the toilet seat system 100, and one lumbar support mechanism 140 and a number of fastening means thereon (e.g., one or more screws, pegs, pins, bolts, velcos, nails, adhesives, etc.) for coupling the lumbar support mechanism 140 to the flange support mechanism 110.

In addition, the toilet seat system 100 may include two arm mechanisms 130A, 130B and a number of fastening means thereon (e.g., one or more screws, velcos, nails, adhesives, etc.) for coupling the two arm mechanisms 130A, 130B to the flange support mechanism 110. Also, as shown in FIG. 2, the toilet seat system 100 may include two bidet-support spring mechanisms 120 and a number of fastening means thereon (e.g., one or more screws, pegs, pins, bolts, velcos, nails, adhesives, etc.) for coupling the two bidet-support spring mechanisms 120 to the flange support mechanism 110 and/or the two arm mechanisms 130A, 130B.

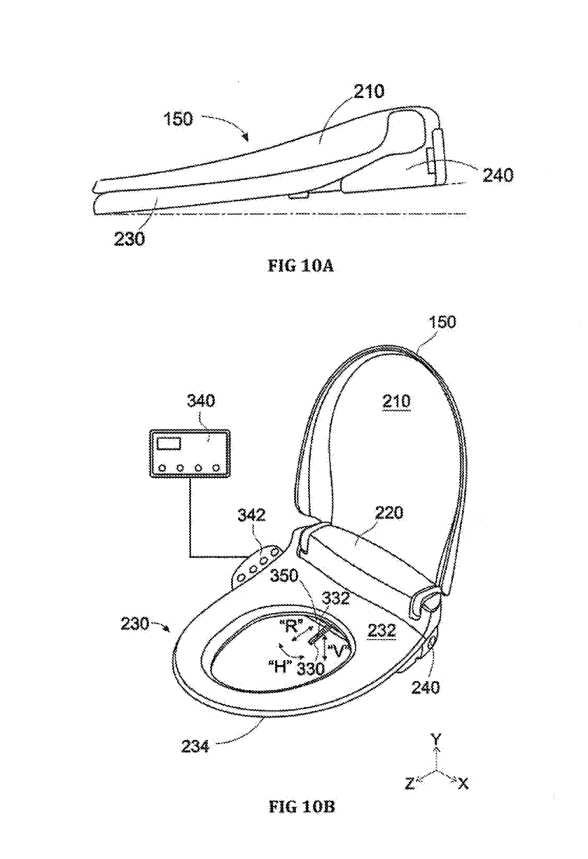

The toilet seat system 100 is designed such that the bidet seat assembly 150 is positioned atop of the bidet-support spring mechanisms 120 and the toilet 160 to support the body of a human subject, such as a bariatric user who uses the toilet seat system 100 for comfort and sufficient weight support. For the purpose of lifting heavy loads of a bariatric user whose weight is much larger than a regular user (e.g., more than 45 kg or higher, such as more than 80 kg, or between about 100 kg to about 400 kg), the bidet-support spring mechanisms 120 are used to support the bidet seat assembly 150 so that the bidet seat assembly 150 is capable of being flexibly movable in vertical direction and support the weight of a bariatric user when the bariatric user is sitting on top of the bidet seat assembly 150 after its cover is opened (the bidet seat assembly 150 is in a closed-cover position in FIG. 1 and FIG. 10A; see also FIG. 10B where the bidet seat assembly 150 is in a opened-cover position). In one example, the one or more bidet-support spring mechanisms 120 are connected by a fastening mean (such as one or more screws, pegs, pins, bolts, nails, velcos, adhesives, etc.) to a bottom of a seat body 240 (shown in FIG. 10B) of the bidet seat assembly 150 and provided to support the bidet seat assembly 150.

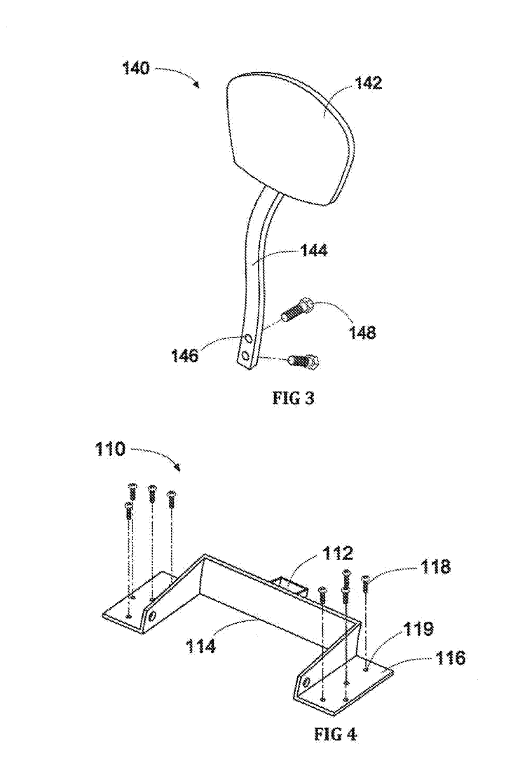

FIG. 3 shows one example of the lumber support mechanism 140, having a back support 142 and a support body 144 according to embodiments of the invention. The support body 144 includes one or more holes 146 for coupling with a number of fastening means (e.g., screws 148, pegs, pins, bolts, nails, velcos, adhesives, etc.) to secure the lumber support mechanism 140 with the flange support mechanism 110.

The back support 142 is shaped curved from top to bottom and from side to side to accommodate the shape of the back of a human user, with many sizes to fit the body width of a human user. The surface of the back portion 142 can be as small as to contact a portion of the back of the user, which makes it possible for a user's back to contact the support portion of the back support during use. The back support 142 may be made of either plastic, wood, steel, etc. and may be formed suitably in the shape of an oval, a square, a rectangular, etc. The lumbar support mechanism 140 may also have a backward-inclinable function. The elastic movement of the back support 142 can be achieved by the deformability or change in the shape of a curved structure of the support body 144.

FIG. 4 shows one example of the flange support mechanism 110, having a flange floor support 116, a support body 114, and a coupling element 112 according to embodiments of the invention. The coupling element 112 can be used to couple the flange support mechanism 110, in one example, to the lumber support mechanism 140 or any other suitable support mechanisms via one or more fastening means (e.g., screws 148, pegs, pins, bolts, nails, velcos, adhesives, etc., as shown in FIG. 3). The flange support mechanism 110 may also include a number of holes (e.g., holes 119) or coupling elements (e.g., one or more fastening means, such as screws 118, pegs, pins, bolts, nails, velcos, adhesives, etc.) for connecting and coupling to the arm mechanisms 130A, 130B and the one or more bidet-support spring mechanisms 120, as shown in FIG. 4.

In FIG. 4, the support body 114 of the flange support mechanism 110 is configured to be mounted to the ground floor via the flange floor support 116 to provide structural support and surround the lower base portion of the toilet 160 of the toile seat system 100. The support body 114 can be made of metal materials, ceramics, stainless steels or plastic materials, etc. and can be formed in reverse U-shape to surround and support the base portion of the toilet 160.

A plurality of screws 118 or other fastening means, can be used to pass through the holes 119 of the flange floor support 116, and then when tightened, mounting the flange support mechanism 110 to the ground floor, which can be a concrete, tiled or wood floor, etc. After the flange support mechanism 110 is mounted on the floor, it is then permanently in place and cannot move upwardly. The toilet 160, being secured to a flange, such as the flange support mechanism 110, is effectively maintained in a rigid position, and without any tendency of the toilet 160 to loosen on the floor when the flange support mechanism 110 is used together with the toilet 160.

As shown in FIG. 4, the coupling element 112 of the flange support mechanism 110 is positioned at the rear side of the support body 114, preferably near the center of the support body 114. In one example, the lower portion of the support body 144 of the lumbar support mechanism 140 can be fixed into the coupling element 112 by one ore more fastening means, such as screws 148.

Accordingly, the lumber support mechanism 140, can be adjusted to a suitable height, such as by providing several holes 146 (as shown in FIG. 3) on the support body 144 and adjusting the length of the support body 144 when it is coupled to the coupling element 112, prior to tightening the screws 148, securing the support body 144 to the coupling element 112, and connecting the lumbar support mechanism 140 to the flange support mechanism 110. Alternatively, the body of the support body 144 can be divided and separated into matching sections that can be interconnected and used to adjust the length of the body of the support body 144 when coupled to the coupling element 112.

During the set up of the toilet seat system 100, the support body 144 of the lumber support mechanism 140 is configured and coupled to the back support 142 on one end, and on the other end inserted onto the coupling element 112. The support body 144 is fixed by fixing screws to the back of the back support 142. The height (the vertical position) of the back support 142 with respect to the height (the vertical position) of the bidet seat assembly 150 is set to a comfortable position for a user sitting on a bidet seat 230 of the bidet seat assembly 150 by adjusting the length of the insertion of the support body 144 into the coupling element 112. After the back support 142 has been set at a suitable height, the screws 148 pass through the holes 146 and the support body 144 and are screwed into the flange support mechanism 110. Prior to using the toilet seat system 100, all these parts as described are assembled and can be readjusted easily.

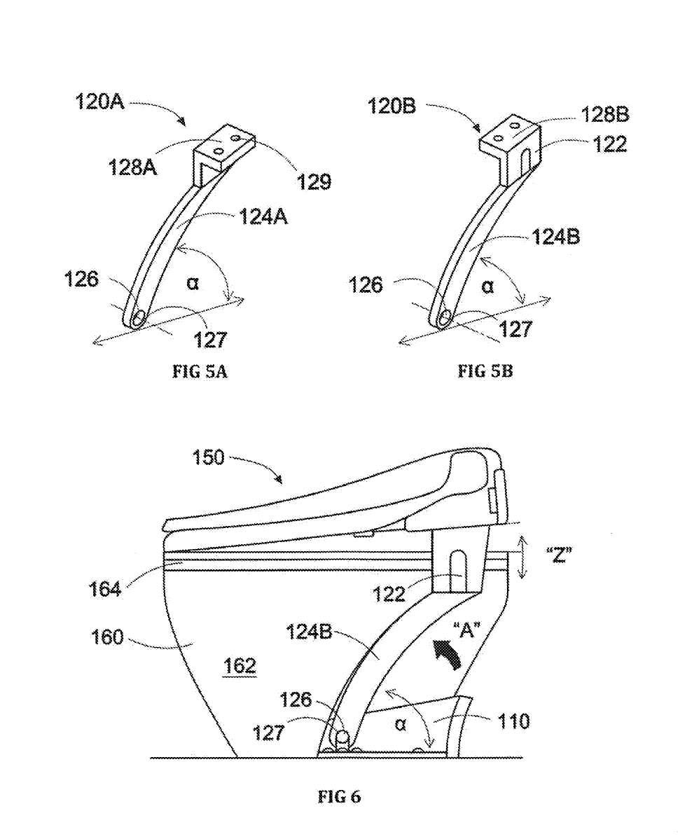

FIGS. 5A-5B and FIG. 6 illustrate examples of the bidet-support spring mechanisms to be coupled to the toilet 160 and the flange support mechanism 110. One or more bidet-support spring mechanisms 120, such as two or more bidet-support spring mechanisms 120A, 120B, can be used to support the bidet seat assembly 150. Once assembled and secured to the flange support mechanism 110 via a number of suitable fastening means (e.g., screws 126, pegs, pins, bolts, nails, velcos, adhesives, etc.), the bidet-support spring mechanisms 120A, 120B are positioned near the right and left sides of the bidet seat assembly 150 for supporting the weights of the bidet seat assembly 150. The toilet 160 may include a toilet bowl 162 and a rim 164.

As shown in FIGS. 5A-5B, the bidet-support spring mechanisms 120A, 120B includes a support body 124A, 124B, respectively. The bidet-support spring mechanisms 120A, 120B may also include one or more attachment elements 128A, 128B wherein their structure are in a shape that can fit into the rim 164 of the toilet seat 160, e.g., in an “L” shape for attaching themselves to (or positioning themselves onto) the rim 164 of the toilet 160. The attachment elements 128A, 128B may also be used to couple and connect the bidet-support spring mechanisms 120A, 120B to the bidet seat assembly 150 via one or more fastening means, holes 129, screw, pegs, pins, bolts, nails, velcos, adhesives, and/or other coupling elements. The support body 124A, 124B may be constructed of any suitable ductile materials, which includes metal materials, structural steel and many alloys of other metals, as characterized by their ability to yield at normal temperatures. The attachment elements 128A, 128B of the upper portion of the bidet-support spring mechanism 120 are positioned on each side of the bidet seat assembly 150.

In one embodiment, one or more springs (such as torsion springs 127 as shown in FIGS. 5A-5B, or other suitable springs) may be coupled to the fastening means (e.g., screws 126, pegs, pins, bolts, nails, velcos, adhesives, etc., which are used to connect the flange support mechanism 110 and the bidet-support spring mechanisms 120A, 120B. A torsion spring is a spring that works by torsion or twisting; that is, a flexible elastic object that stores mechanical energy when it is twisted or in helical structure. When it is twisted, it exerts a force (actually torque) in the opposite direction, proportional to the amount (angle) it is twisted.

FIG. 6 is a side view of the toilet seat assembly 150 positioned atop the bidet-support spring mechanism 120 and the toilet 160, according to one embodiment of the invention, showing the configuration of the support body 124A in a curved position in an angle “a”, the spring 122, and the spring 127 so that the bidet-support spring mechanism 120 is adapted to be flexibly moving up and down in a vertical direction “Z”, such that the support body 120 can move flexibly in a vertical plane “A”, according to one or more embodiments of the invention.

In another embodiment, the support body 124A, 124B are positioned vertically in an angle “α, as compared to a ground plane of the toilet seat system 100 so that the whole structure of the bidet-support spring mechanisms 120A, 120B, together with the assistance of the torsion springs 127, is adapted to swivel flexibly in a vertical plane “A”, leading to the attachment elements 128A, 128B to be able to move flexibly up and down in a vertical “Z” position (as shown in FIG. 6), which is advantageous when the attachment elements 128A, 128B are attached to the bidet seat assembly 150 such that the bidet seat assembly 150 is then adapted to move flexibly up and down in a vertical “Z” direction in supporting the weight of a bariatric user (or a regular user).

In still another embodiment, the bidet-support spring mechanisms 120A, 120B additionally include one ore more springs 122 positioned inside the attachment elements 128A, 128B. Examples of the springs 122 include compression springs, coiled wires, conventional coils, etc., and can be constructed of metal materials and other suitable elastic materials, such as spring steel, brass, stainless steel materials or other suitable materials. The configuration of the springs 122 are adapted to support and sustain the heavy-weight of a bariatric user when the bariatric user is using the toilet seat system 100 and sitting on tope of the bidet seat assembly 150.

A plurality of spring can be arranged generally parallel to one another and transversely bridge in a uniform manner to provide a spring effect when a downward pressure is applied thereto. The springs 122 are used to provide a relationship of constant load or force over a limited range of deflection. When a person is sitting at the bidet seat assembly 150, the springs 122 are compressed from their resting positions, and exert an opposing force approximately proportional to the change in their lengths. Usually the effective force (as a resulting of the weight of the user sitting on the bidet seat assembly 150) exerted by the springs varies considerably over the entire deflection range. The bidet-support spring mechanism 120 may also minimize the effect of shifting loads applied to the seat area of the bidet seat assembly 150.

In one example, the attachment elements 128A, 128B of the bidet-support spring mechanism 120 can touch or contact the upper portion of the rim 164 (as shown in FIG. 6) when a user is using the toilet seat system 100 or when the bidet-seat assembly 150 is in an open-cover position. In another example, the attachment elements 128A, 128B of the bidet-support spring mechanism 120A, 120B do not touch or contact the upper portion of the rim 164 when a user is not using the toilet seat system or when the bidet-seat assembly 150 is in a closed-cover position.

The construction of the support body 124A, 124B in a flexible angled position, the use of the torsion springs 127, the use of the springs 122 within the attachment elements 128A, 128B, or the combinations thereof, enables the bidet seat assembly to be able to move flexibly up and down in a vertical position in supporting the weight of a bariatric user (or a regular user). Not wishing to be bound by theory, it is contemplated that the weight of an user sitting on the top of the bidet seat assembly 150 of the toilet seat system 100 of the invention is then evenly distributed to the one or more bidet-support spring mechanisms 120A, 120B and, via the bidet-support spring mechanisms 120A, which is connected to the flange support mechanism 110, 120B; thus the weight is ultimately distributed to the flange support mechanism 110. Further, the use of the torsion springs 127 and/or the use of the springs 122 help to absorb a portion of the weight of the user, especially for a bariatric user.

The bidet-support spring mechanism 120 is accordingly adapted to provide better weight distribution for the toilet seat system 100 and security for a user-friendly and comfortable toilet experience. When a heavy-weight bariatric person is sitting on the toilet seat system 100, the spring mechanism 120, being slightly tilted in an “α [symbol] angle and with the help of the spring 122 and the torsion spring 127 provides uniform weight pressure and structural support, transmitting via the bidet-support spring mechanism 120 and the flange support mechanism 110 to the ground floor. The bidet-support spring mechanism 120 generally offers firmer support within the seating area and in addition provides a wider weight capability for the toilet seat system 100 to accommodate a regular user to a heavy-weight user.

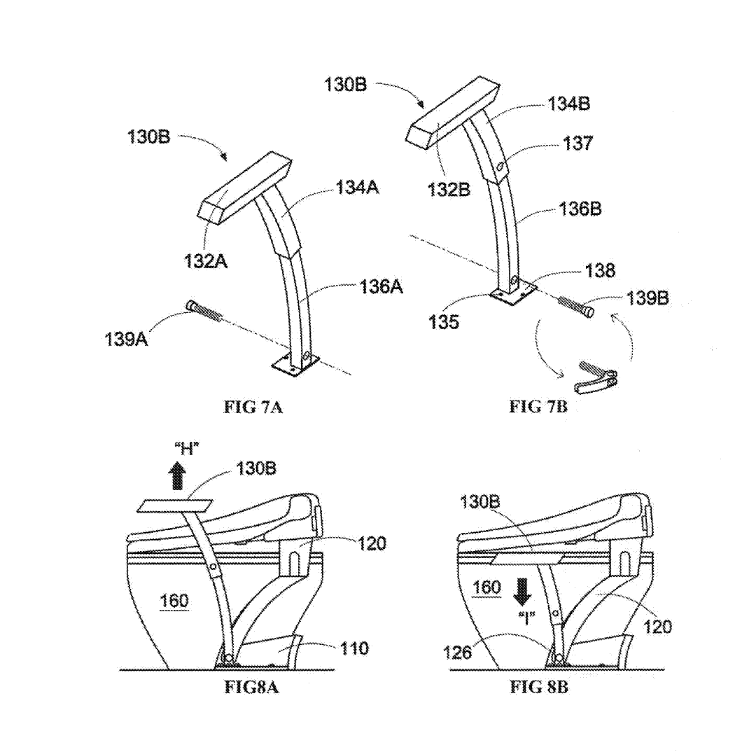

FIGS. 7A-7B shows examples of the arm mechanisms 130A, 130B, respectively, having an armrest 132A, 132B, an upper support body 134A, 134B, a lower support body, 136A, 136B according to one or more embodiments of the invention. Each of the upper support bodies 134A, 134B of the arm mechanisms 130A, 130B can be connected to the lower support bodies 136A, 136B via a fastening element 137. The type of the fastening element 137 that can be used is not limited, and examples of the fastening element 137 includes pegs, pins, screws, bolts, etc., among others.

In FIG. 7A, the arm mechanism 130A is a retractable arm mechanism and includes the armrest 132A, the upper support body 134A, and the lower support body 136A. In FIG. 7B, the arm mechanism 130B is a retractable arm mechanism and includes the armrest 132B, the upper support body 134B, and the lower support body 136B. In one or more examples, each of the upper support bodies 134A, 134B can have a circular or rectangular cross-section with an inner diameter a little bit larger than the outer diameter of each of the lower support bodies 136A, 136B, or vice versa. Each of the components and parts of the arm mechanisms 130A, 130B can be made of molded plastic, plywood, hard rubber, or even stainless steel.

In addition, the use of the upper support bodies 134A, 134B, and the lower support bodies, 136A, 136B allows the arm mechanisms 130A, 130B to be in the form of two or more length and configurations. In a first configuration, the arm mechanisms 130A, 130B can be at a full length (also shown in FIG. 8A), in which case each of the upper support bodies 134A, 134B extends upwardly up to a first terminal point, which may be the highest in vertical “Z” direction. In a second configuration, the arm mechanisms 130A, 130B can be at a shortest length (also shown in FIG. 8B), in which case each of the upper support bodies 134A, 134B extends downwardly to a second terminal point, which may be the lowest in vertical “Z” direction.

Accordingly, the upper support bodies 134A, 134B form as upward extensions of the lower support bodies, 136A, 136B and have a geometric and aesthetic continuity with respect to the lower support bodies, 136A, 136B. Each of the fastening elements 137 is fitted onto the upper support body 134A (or the upper support body 134B) so that the support bodies of the arm mechanism 130 can be extended to have different length and as a result, the armrest 132A, 132B can be adjusted vertically up and down to reach the desired height for different people by loosing and tightening of the fastening element 137 and adjust the upper support bodies, 134A, 134B to a different vertical positions, as compared to the positions of the lower support bodies, 136A, 136B.

Each of the arm mechanisms 130A, 130B are secured to a flange 138 or other securing mechanism located on the ground by a number of coupled fastening mechanism, including holes and screws 139A, 139B, etc. The fastening elements 139A, 139B can be any key hole fasteners, screws, bolts, other connectors or combinations thereof. The arm mechanisms 130A, 130B can also be mounted onto the flange support mechanism 110, as shown in FIGS. 8A-8B. In one or more examples, the arm mechanisms 130A, 130B are mounted onto the flange support mechanism 110. Once being assembled and secured to the flange support mechanism 110 via a number of suitable fastening means, the one or more arm mechanisms 130A, 130B are positioned near the bidet seat assembly 150 for supporting the right and left arms and weights of a bariatric user, e.g., the user 170, so that the bariatric user can use the bidet seat assembly with ease.

FIG. 8A and FIG. 8B provide sides view of two exemplary bariatric toilet seat systems where the armrest 132A, 132B of the arm mechanism 130A, 130B of the toilet seat system 100 are adapted to be moving up and down in a direction “H”, as shown in FIG. 8A, and in a direction “I”, as shown in FIG. 8B, respectively, when the arm mechanisms 130A, 130B. In the example of FIG. 8A, the toilet seat system 100 is not being pressed with a force, or is leveled with an up force. In the example of FIG. 8B, the toilet seat system 100 is being pressed with a down force, or is leveled with a down force.

In one or more embodiment, the upper support bodies 134A, 134B and/or the lower support bodies, 136A may be curved in shape such that the arm mechanism 130A, 130B provide better upward and downward springing weight support for the arms of heavy weight users. In addition, the adjustment of the heights of the armrests 132A, 132B is performed by sliding the upper support bodies 134A, 134B along the lower support bodies 136A, 136B. In another embodiments, the heights of the armrests 132A, 132B can be at fixed positions by tightening the fastening element 137. When the adjusting the height of the upper support bodies 134A, 134B, the fastening element 137 is loosened until the upper support bodies 134A, 134B are sliding along the lower support bodies 136A, 136B and being adjusted to a desired height, and then the fastening element 137 can be tightened.

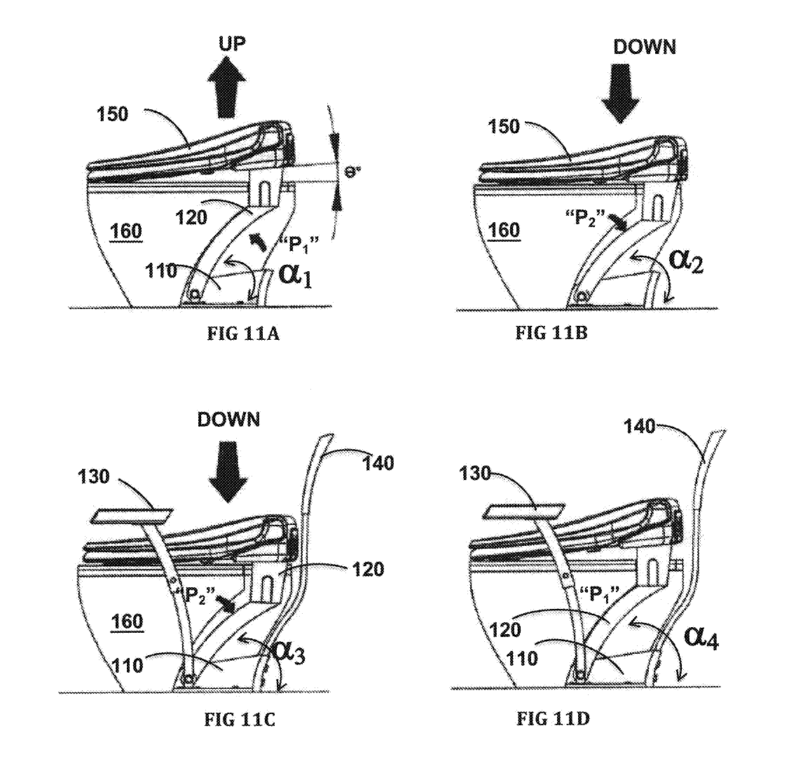

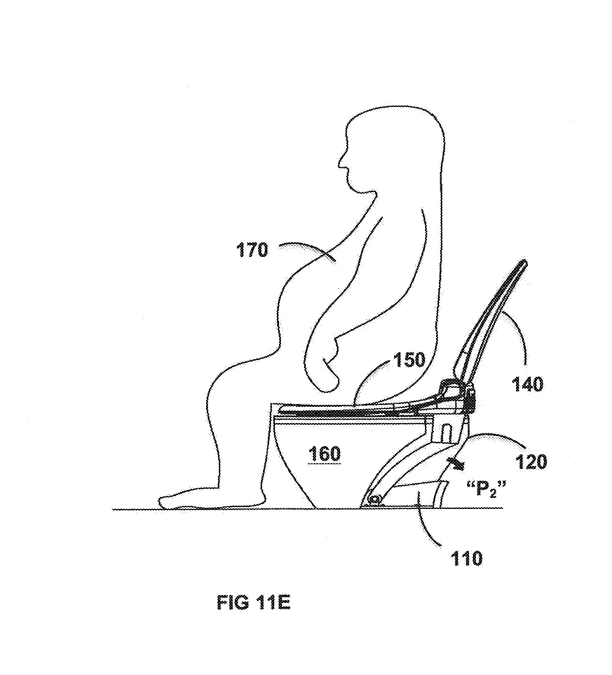

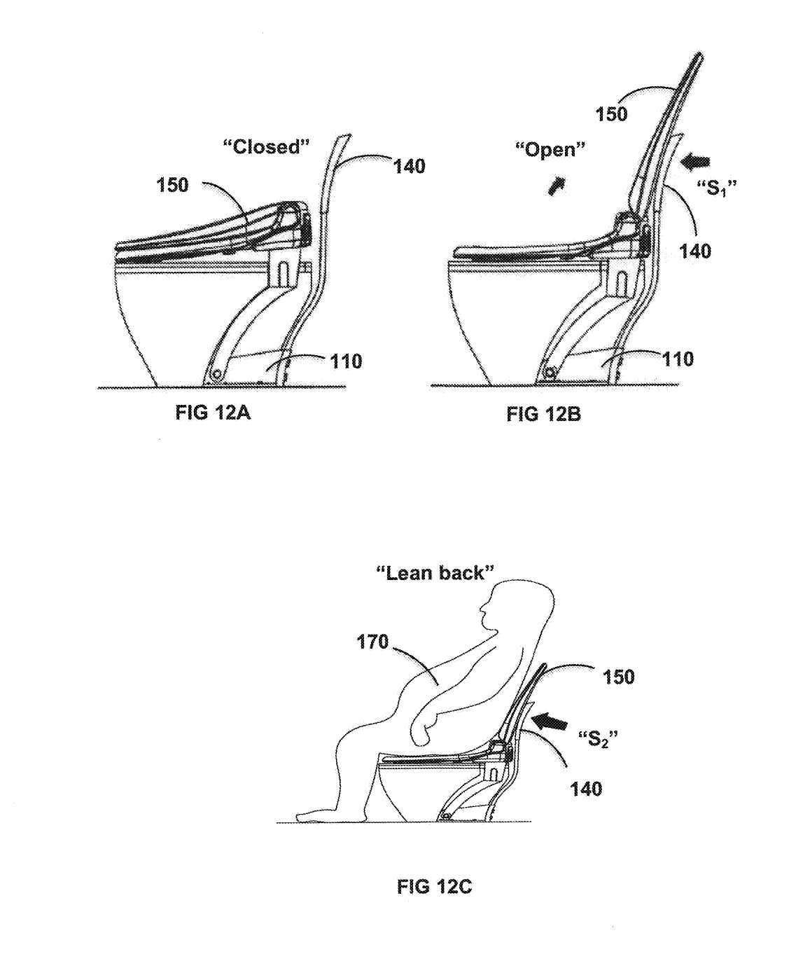

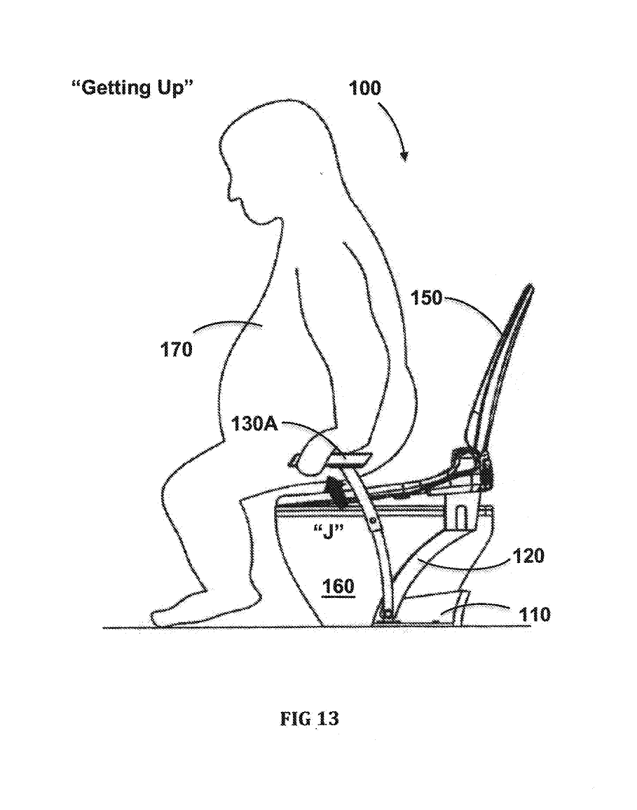

As shown in FIGS. 8A and 8B, the bidet-support spring mechanism 120 is suitable to be positioned under the toilet seat assembly 150 to receive the weight of a user 170, (e.g., a human a shown in FIG. 11E, FIG. 12C and FIG. 13) so that the weight of the user 170 is distributed onto the bidet-support spring mechanism 120 and relieved from the bidet seat assembly 150 positioned on top of the toilet 160. In addition, the bidet seat assembly 150 has large area on its seat portion and is positioned at a height that is ergonomic to the user 170.

In the examples of FIGS. 8A-8B, both the arm mechanisms 130A, 130B and the bidet-support spring mechanism 120 can be assembled together and also be secured together onto the flange support mechanism 110. In other examples, the arm mechanisms 130A, 130B can be secured to the flange support mechanism 110 in a separate location different from the bidet-support spring mechanism 120. In addition, the arm mechanisms 130A, 130B can be secured to the ground in the same or a separate location different from the bidet-support spring mechanism 120.

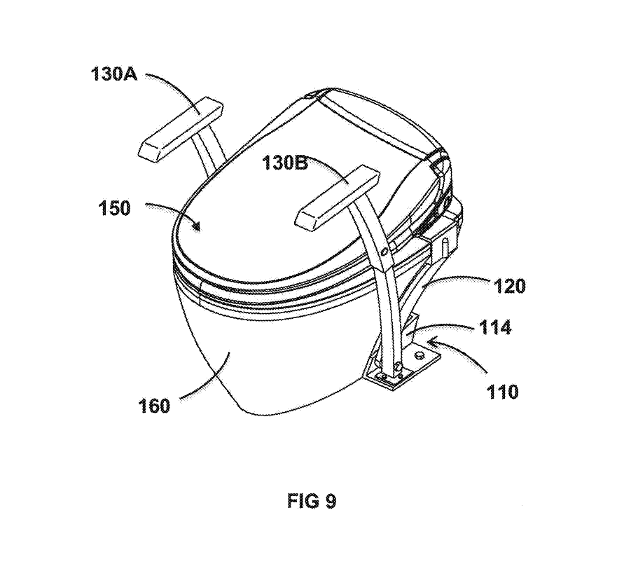

The arm mechanisms 130A, 130B can be connected together with the bidet-support spring mechanism 120 and the flange support mechanism 110 by a number of fastening elements, which can be any key hole fasteners, the screws 126, bolts, other connectors or combinations thereof. key hole fasteners, the screws 139A, 139B, or other connectors passing through holes. In one embodiment, via securing to the same fastening mechanism (e.g., the screws 126, or any other screws, bolts, etc.) the toilet seat system 100 may have the arm mechanisms 130A, 130B that is in close contact with and connected to the outer surfaces of the bidet-support spring mechanism 120m, and the spring mechanism 120 that is in close contact with and connected to the flange support mechanism 110. The arm mechanisms 130A, 130B can be mounted on the flange support mechanism 110 to the floor by one or more screws. Accordingly, the toilet seat system 100 as described herein can be used for obese people to allow them with a comfortable user experience, and is suitable in any hospitals or medical installations and homes. FIG. 9 provides a perspective view of an exemplary bariatric toilet seat systems when the arm mechanism 130A, 130B and the bidet-support mechanism 120 are assembled to the top of the bidet seat assembly 150.

FIG. 10A is a side view of one example of the bidet seat assembly 150, shown with a seat cover 210 covering a bidet seat 230 above a base 240 in a closed position, according to embodiments of the invention. The bidet seat assembly 150 can be placed on top of a traditionally toilet bowel (e.g., the toilet 160, etc). In one example, the bidet seat assembly 150 can be installed to a toilet system by removing any existing seat from a traditional toilet system and replacing a traditional seat with the seat assembly 150. In one example, the bidet seat assembly 150 may have fastening elements (e.g., screws bolts, velcos, etc.) in the back to be connected to a traditional toilet bowel.

FIG. 10B is a perspective view of the bidet seat assembly 150 with the seat cover 210 being positioned in an open position. The bidet seat assembly 150 may include the seat cover 210, a base housing 220, the bidet seat 230, the base 240 (optionally, having a medicine delivery assembly therein), one or more drying assemblies 350, one or more spray nozzle assemblies 330 with one or more spraying nozzles 332, the base 240, a remote control unit 340, and optionally a control unit 342 adjacent the bidet seat 230, according to embodiments of the invention.

The seat cover 210 is generally closed to cover the base 240. During operation or in idle position, the seat cover can be opened and positioned upward, such as being positioned against a water tank. The seat 230 includes the seat body 232 facing upward so that a human subject can sit thereon. The base housing 220 and the base 240 are formed to connect the seat cover 210 and the seat 230 together, for example, using one or more hinges such that the seat cover 210 can be opened or closed against the bidet seat 230. The base housing 220 and the base 240 are used generally to house one or more assemblies, e.g., the drying assembly 350, the spray nozzle assembly 330, and a medicine delivery assembly, etc., therein. In one example, the spray nozzle assembly 330 is positioned inside the base housing 220 and exposed to be seen from the bottom of the seat body 232 adjacent the base housing 220.

The spray nozzle assembly 330, and one or more medicine delivery assemblies can be used for delivering and applying water, cleaning solutions, and/or medicines to a region of a human body that may not otherwise be easily accessible (e.g., to the perianal region), and washing the region of the human body, as well as drying the region using the drying assembly 350, drying the region of the human body. In addition, a method of operating the toilet seat system 100 is also provided.

In one embodiment, the bidet seat assembly 150 as described herein may include a remote-controlled medicine delivery assembly having one or more spraying nozzles 332, a first motor being connected to the spraying nozzles 332 and capable of directing one or more extending and retracting movements of the spraying nozzles 332, one or more second motors being connected to the spraying nozzles 332 and capable of directing one or more three-dimensional rotational movements of the spraying nozzles 332, and one or more dynamic steering remote control units adapted to be communicating with both the first motor and the one or more second motors and directing one or more movements of the first motor and the one or more second motors.

In another embodiment, the spray nozzle assembly 330 includes a wash nozzle and a medicinal delivery nozzle. In one example, the wash nozzle may include a water jet head. In another example, the medicinal delivery nozzle includes a medicinal liquid inlet being connected to a medicinal storage assembly and adapted for jetting out a medicine-containing solution. In addition, the medicinal storage assembly includes one or more cartridges that store medicines to be applied, and a pressurized pump adapted to pump and deliver the medicines from the one or more cartridges to the medicinal liquid inlet. The medicinal delivery nozzle may further include a medicinal atomizer head adapted to modify a medicine to achieve a consistency that can be applied over a distance and deliver a stream of the medicine to a user. In one aspect, the medicinal delivery nozzle is adapted to deliver one or more medicines across a space to a localized region of a body of a user. One example of the localized region is a perianal region of the body of the user.

In still another embodiment, the spray nozzle assembly 330 provided herein includes one or more dynamic steering remote control units that receive an user input to direct the one or more movements of a first motor and one or more second motors and adjust at least one of an angle or a location of the delivery nozzle assembly based on the user input. In still another embodiment, the spray nozzle assembly 330 is remote-controlled and coupled to an base unit and adapted to receive a perianal region of a user. Examples of the base unit include, but are not limiting to, a bidet toilet seat washing system, a bidet medicine delivery seat, a toilet seat assembly, a bedpan, a hydraulic chair, a commode, a hospital bed, among others. In one example, the base unit of the remoted-controlled medicine delivery assembly is a toilet seat assembly, which contains a housing and a base seat.

Additional embodiments of the invention provide one or more methods of operating and/or using remote-control units to control the movements of the toilet seat system 100. The method includes controlling one or more movements of a spray nozzle assembly by one or more dynamic steering remote control units. In one aspect, the one or more movements of a spray nozzle assembly is controlled by one or more dynamic steering remote control units remotely via wireless means or long-wired means.

The remote control units 340, 342 are adapted to be communicating and directing one or more movements of the one or more assemblies, such as the one or more drying assemblies 350, the one or more spray nozzle assemblies 330 with one or more spraying nozzles 332, and one or more medicine delivery assemblies. The one or more spraying nozzles 332 of the spray nozzle assembly 330 are able to jet water or any liquid to a localized region (e.g. perianal region) of a human body private part. In one example, the spray nozzle assembly 330 are able to move in a direction marked as “R” to be extended and retracted in and out of the base housing 220. In another example, the one or more spraying nozzles 332 of the spray nozzle assembly 330 are able to move in a direction marked as “V” to move up and down (e.g., in a Z-direction or a gravitational direction), particularly after the spray nozzle mechanism 330 are extended and retracted out. In another example, the spray nozzle mechanism 330 are able to move in a direction marked as “H” to move left-right, particularly after the spray nozzle mechanism 330 are extended and retracted out.

The remote control units 340, 342 can cause the user 170 to operate the operation button reliably. The remote control units 340, 342 thus provide “hands-free” options, therefore avoiding some discomfort or embarrassment from the user 170. For example, one or more control units 340 can be mounted on the wall adjacent to or far away from the toilet 160. The control unit 340 has a screen and a keyboard with pushbuttons for turning the water or air on and off, for controlling the drying air temperature, etc. The water flow level may be selected by appropriate command through the remote control unit. The remote control unit is used to transit appliance control data to the toilet bidet system 100. For example, the toilet bidet system 100 may include a decoder to decode transmitted data. When the data is decoded, the toilet bidet 100 responds to an appliance control signal suitable and control the movements of the one or more assemblies inside the bidet seat assembly 150. For another example, the control units 342 can be mounted on one or more sides of the base 240. It enables a user to directly control the toilet bidet system 100.

FIGS. 11A-11E are side views of the toilet seat system 100 where the bidet seat assembly 150 is designed to be movable up and down in the vertical “Z” direction atop of the toilet 160, according to embodiments of the invention. As shown in FIG. 11A, when the toilet seat system 100 is not loaded with any force, the bidet seat assembly 150 is atop of the toilet 160 at an angle θ°, such that the spring 122 inside the bidet-support spring mechanism 120 is in its relaxed configuration. There is also no deformation or change in its original curved shape in the support bodies 124A, 124B. The angle θ° between the bottom surface of the bidet seat 150 and the top surface of the toilet 160 is larger than zero degree and may be at 5° to 45° or larger.

In FIG. 11A, while the bidet-support spring mechanisms 120A, 120B are in its relaxed configuration, the support body 124A, 124B of the bidet-support spring mechanisms 120A, 120B are curved in an angle α₁, which extends vertically from zero degree to less than 90°. For example, the angle α₁ may be angled at about 45°.

As shown in FIG. 11B, when the toilet seat system 100 is loaded with a force (e.g., the weight of a bariatric user as exerted as a force “P₂”) to push the bidet assembly 150 downwardly. When being loaded with a large enough force such as a heavy weight of a human user, the bidet seat assembly 150 may be moved downwardly to be directly in contact with the top surface of the toilet 160. As a result, when the bidet assembly 150 is pushed down to be in close contact with the top surface of the toilet 160, the spring 122 inside the bidet-support spring mechanism 120 is in its tightly compressed configuration and the angle θ° is equal to zero degree. In addition, the support body 124A, 124B of the bidet-support spring mechanisms 120A, 120B are curved in an angle α₂, which extends vertically from zero degree to less than 90°. For example, the angle α₂ may be angled at less than about 45°. In one embodiment, the angle α₂ is smaller than the angle α₁ since the bidet-support spring mechanisms 120A, 120B are being pushed downwardly.

The spring 122 may exert an opposing force (e.g., a force “P₁” as shown in FIG. 11A) which is in equal and opposite direction of the force (e.g., the direction of the weight of a human user) that's compressing the spring and thus resisting and supporting the weight of the human user. The opposing force a spring exerts follow Hooke's law, and is related with spring constant which measures how strong the spring is and the distance the spring is compressed in its compressed configuration/state, different from it equilibrium or rest configuration/state.

As shown in FIG. 11C, when an user of a heavy weight sitting on the bidet seat assembly 150, it transferred the weight as an applied force (e.g., a force “P₂” as shown in FIG. 11C) to the bidet-support spring mechanism 120 and produces a stress, thus an opposing force (e.g., a force “P₁” as shown in FIG. 11C), which then causes the spring 122 to deform and the bidet-support spring mechanism 120 to flexibly bend and support the weight of the user. As deformation of the spring 122 occurs, internal inter-molecular forces arise and oppose the applied force (e.g., a force “P₁” as shown in FIG. 11D). This is a temporary shape change, which is self-reversing after the force is removed, so that the spring 122 and the bidet-support spring mechanism 120 returns to its original shape. If the spring 122 and the bidet-support spring mechanism 120 are given an overload exceeding a yield point, plastic deformation is caused. This type of deformation is irreversible. While this type of locking system is capable of resisting a heavy weight user (e.g., a weight of approximately 200 pounds or larger, such as 300 pounds or larger or 400 pounds or larger) because of the selected spring and the deformation described hereinabove, this is quite adequate to securely support the weight of an obese user.

In FIG. 11C, the support body 124A, 124B of the bidet-support spring mechanisms 120A, 120B are curved in an angle α₃, which extends vertically from zero degree to less than 90°. For example, the angle α₃ may be angled at less than about 45°. In FIG. 11D, the support body 124A, 124B of the bidet-support spring mechanisms 120A, 120B are curved in an angle α₄, which extends vertically from zero degree to less than 90°. For example, the angle α₄ may be angled at about 45°. In one embodiment, the angle 3₂ is smaller than the angle α₄.

FIG. 11E illustrates one example when a human subject is sitting on top of the bidet seat assembly and using a bariatric bidet toilet seat system with the bidet seat assembly 150, the bidet-support spring mechanism 120, and, according to one or more embodiments of the invention. As shown in FIGS. 11A-11E, the weight of an obese user can be supported by the opposing force (e.g., a force “P₂” as shown in FIG. 11E) as exerted by the spring 122 and the bidet-support spring mechanism 120 to support the user sitting atop the bidet seat assembly 150. In addition, a portion of the applied force exerted by the weight of an obese user can also be supported by the lumber support mechanism 140 once the obese user is in contact with the lumber support mechanism 140.

FIGS. 12A-12C are side view of examples of a bariatric bidet toilet seat system (e.g., the toilet seat system 100) having the lumber support mechanism 140, and the bidet seat assembly 150, where the seat cover 210 of the bidet seat assembly 150 is closed, as shown in FIG. 12A, or opened, as shown in FIG. 12B, and can be pressed against a back support of the lumber support mechanism 140 so that the back support of the lumber support mechanism 140 is in a sit-down “S₁” position, according to one or more embodiments of the invention. When the bidet toilet system 100 is not used, the seat cover 210 is closed to cover the base 240. When the bidet seat assembly 150 is not loaded with a force, there is no deformation of the spring 122, and the spring 122 is in its equilibrium position. There is also no deformation or change in the curved shape of the support bodies 124A, 124B and the lumbar support mechanism 140. During operation, the seat cover 210 can be opened and positioned upwardly and a bariatric user can sit down on the top surface of the bidet seat 230 and use and operate the bidet seat assembly 150. As the user is sit down, each or the combination of the compression of the spring 122, and the arched deformation of the shape and mechanical strength of the support bodies 124A, 124B and the lumbar support mechanism 140 helps to support the weight of the bariatric user.

FIG. 12C is a side view of another example of the bidet toilet seat system 100, where a cover of the bidet seat assembly 150 is opened and a bariatric user is sitting down and positioned atop of the bidet seat assembly. In this example, the bariatric user is able to comfortably lean backward against the back support of the lumber support mechanism 140 so that the back support of the lumber support mechanism 140 is in a sit-down “S₂” position, according to one or more embodiments of the invention. The lumber support mechanism 140, the bidet support spring mechanism 120 and the spring 122 can functions together to support the heavy weight of a bariatric user. Prolonged sitting in a bidet seat system is frequently attended by discomfort in the back of the user. Actually, it commonly is recognized that rapid tiredness is encountered during sitting. The region of discomfort varies from persons to persons. This discomfort may derive from a particular configuration, construction and the back support of a seat system being used. The toilet seat system 100 with the lumbar support mechanism 140 light in weight, pleasing in appearance, and readily secured or adjusted provides a direct and effective manner to closely satisfy the back support requirements of each individual user.

FIG. 13 illustrates one example when the bariatric user is getting up after sitting atop of the bidet seat assembly 150 and using a toilet bariatric bidet toilet seat system. In this example, the bariatric user can use one or both of his hands to exert pressure and weight and press down the armrests 132A, 132B of the arm mechanisms 130A, 130B, such that the weight of the bariatric user is supported by the arched shape and mechanical strength of the arm mechanisms 130A, 130B, which ultimately is supported by the ground where the arm mechanisms 130A, 130B are connected to.

While being seated on the toilet seat system 100 equipped with the arm mechanisms 130A, 130B, the human user can rest his or her arms on the armrests 132A, 132B comfortably, and when standing up from the bidet seat system 100, the human subject can press his or her hands against the armrests 132A, 132B, exerting his or her weight on the armrest 132A, 132B, and being supporting for an opposing force, as shown in FIG. 13 as a force, “J”, being the mechanical strength of the arm mechanisms 130A, 130B and ground floor support. The human user can prop his or her upper body up with the help of his or her hands in such a way as to stand up more easily. The toilet seat system 100 equipped with adjustable armrests 132A, 132B described herein provides more comfortable and convenient user experience to each user.

Accordingly, the invention provides the toilet seat system 100 for supporting a body of a bariatric user, where one or more bidet-support spring mechanisms 120 are positioned adjacent to a toilet, and the bidet seat assembly 150 are positioned atop the one or more bidet-support spring mechanisms 120 and the toilet 160 to support the body and weight of the human user. The one or more bidet-support spring mechanisms 120 are connected to a bottom of a seat body 232 of the bidet seat assembly 150 and provided to support the bidet seat assembly 150 and thus the weight of the human user sitting atop the bidet seat assembly 150 so that the bidet seat assembly is capable of being flexibly movable in vertical direction. In one embodiment, the bidet seat assembly 150 includes the spray nozzle assembly 330 for jetting out water at a temperature ranging from between room temperature to about 90° C. In addition, the flange support mechanism 110 is connected to the one or more bidet-support spring mechanisms 120 and being secured to the floor where the toilet seat system 100 is positioned thereon. The bariatric toilet seat system can be automated with a user-friendly control system built inside the bariatric toilet seat system and controlled by an attached control unit or a remote control. Methods of forming the bariatric toilet seat system and methods using the bariatric toilet seat system are also disclosed.

While the foregoing is directed to embodiments of the present invention, other and further embodiments of the invention may be devised without departing from the basic scope thereof, and the scope thereof is determined by the claims that follow.

1. A toilet seat system for supporting a body of a human subject, comprising:
a toilet;
one or more bidet-support spring mechanisms positioned adjacent to the toilet; and
a bidet seat assembly being positioned atop the one or more bidet-support spring mechanisms and the toilet to support the body of the human subject, wherein the one or more bidet-support spring mechanisms are connected to a bottom of a seat body of the bidet seat assembly and provided to support the bidet seat assembly so that the bidet seat assembly is capable of being flexibly movable in vertical direction.

2. The toilet seat system of claim 1, wherein the bidet seat assembly comprises a drying assembly for blowing out air at a temperature ranging from between room temperature to about 90 degree Celsius.

3. The toilet seat system of claim 1, wherein the bidet seat assembly comprises a spray nozzle assembly for spraying out water at a temperature ranging from between room temperature to about 90 degree Celsius.

4. The toilet seat system of claim 3, wherein the spray nozzle assembly further comprises:
a spraying nozzle;
a first motor being connected to the spraying nozzle of the spray nozzle assembly, the first motor being capable of directing one or more extending and retracting movements of the spraying nozzle of the spray nozzle assembly;
one or more second motors being connected to the spraying nozzle of the spray nozzle assembly and capable of directing one or more three-dimensional rotational movements of the spraying nozzle of the spray nozzle assembly; and
one or more dynamic steering remote control units adapted to be communicating with both the first motor and the one or more second motors and directing one or more movements of the first motor and the one or more second motors.

5. The toilet seat system of claim 1, wherein the bidet seat assembly comprises a medicine delivery assembly for delivering medicine to the body of the human subject.

6. The toilet seat system of claim 1, wherein the one or more bidet-support spring mechanisms comprises an attachment element to be attached to a rim of a toilet bowl of the toilet.

7. The toilet seat system of claim 1, wherein the one or more bidet-support spring mechanisms further comprises a spring connected to the attachment element so that the attachment element is flexibly movable in vertical direction to support the bidet seat assembly.

8. The toilet seat system of claim 7, wherein the one or more bidet-support spring mechanisms further comprises a support body, which is capable of being curved in an angle α, which extends vertically from zero degree to less than 90°.

9. The toilet seat system of claim 1, further comprising:
a flange support mechanism connected to the one or more bidet-support spring mechanisms and being secured to a floor where the toilet seat system is positioned thereon.

10. The toilet seat system of claim 9, wherein the flange support mechanism comprises a support body and a flange floor support being secure to the floor by a number of screws.

11. The toilet seat system of claim 9, wherein the flange support mechanism comprises a coupling element to couple a lumber support mechanism to the flange support mechanism.

12. The toilet seat system of claim 1, further comprising:
a lumber support mechanism comprising a back support, a support body, wherein the arm is coupled to a flange support mechanism.

13. The toilet seat system of claim 1, further comprising:
one or more arm mechanisms, each arm mechanism comprises an armrest, one or more support bodies, each armrest is provided to support an arm of the human subject.

14. The toilet seat system of claim 13, wherein the one or more arm mechanisms are coupled to a flange support mechanism.

15. A toilet seat system for supporting a body of a human subject, comprising:
a toilet;
one or more bidet-support spring mechanisms positioned adjacent to the toilet; a flange support mechanism connected to the one or more bidet-support spring mechanisms and being secured to a floor where the toilet seat system is positioned thereon; and
a bidet seat assembly being positioned atop the one or more bidet-support spring mechanisms and the toilet to support the body of the human subject, wherein the one or more bidet-support spring mechanisms is connected to a bottom of a seat body of the bidet seat assembly and provided to support the bidet seat assembly so that the bidet seat assembly is capable of being flexibly movable in vertical direction.

16. The toilet seat system of claim 15, wherein the bidet seat assembly comprises a spray nozzle assembly for spraying out water at a temperature ranging from between room temperature to about 90° C.

17. The toilet seat system of claim 15, wherein the flange support mechanism comprises a support body and a flange floor support being secure to the floor by a number of screws.

18. The toilet seat system of claim 15, wherein the flange support mechanism is coupled to a lumber support mechanism.

19. The toilet seat system of claim 18, wherein the flange support mechanism and the lumber support mechanism are coupled by a coupling element of the flange support mechanism.

20. The toilet seat system of claim 15, wherein the flange support mechanism is coupled to one or more arm mechanisms, wherein each of the one or more arm mechanisms comprises one or more support bodies and an armrest, each armrest is provided to support an arm of the human subject.

21. A toilet seat system for supporting a body of a human subject, comprising:
a toilet;
one or more bidet-support spring mechanisms positioned adjacent to the toilet; a flange support mechanism connected to the one or more bidet-support spring mechanisms and being secured to a floor where the toilet seat system is positioned thereon; and
a bidet seat assembly being positioned atop the one or more bidet-support spring mechanisms and the toilet to support the body of the human subject,
wherein the one or more bidet-support spring mechanisms is connected to a bottom of a seat body of the bidet seat assembly and provided to support the bidet seat assembly so that the bidet seat assembly is capable of being flexibly movable in vertical direction, and
wherein the bidet seat assembly comprises a spray nozzle assembly for jetting out water at a temperature ranging from between room temperature to about 90° C.

22. The toilet seat system of claim 21, wherein the bidet seat assembly further comprises a drying assembly for blowing out air at a temperature ranging from between room temperature to about 90° C.

23. The toilet seat system of claim 21, wherein the bidet seat assembly further comprises a medicine delivery assembly for delivering medicine to the body of the human subject.

24. The toilet seat system of claim 21, wherein the spray nozzle assembly further comprises:
a spraying nozzle; a first motor being connected to the spraying nozzle of the spray nozzle assembly, the first motor being capable of directing one or more extending and retracting movements of the spraying nozzle of the spray nozzle assembly;
one or more second motors being connected to the spraying nozzle of the spray nozzle assembly and capable of directing one or more three-dimensional rotational movements of the spraying nozzle of the spray nozzle assembly; and
one or more dynamic steering remote control units adapted to be communicating with both the first motor and the one or more second motors and directing one or more movements of the first motor and the one or more second motors.
